(19) United States
(12) Patent Application Publication
(10) Pub. No.: US 2010/0189819 A1
(43) Pub. Date: Jul. 29, 2010

(54) NEURO-DEGENERATIVE INHIBITOR, NEURO-ENDOCRINE MODULATOR, AND NEURO-CEREBRAL METABOLISM ENHANCER

(75) Inventor: Nathan SASSOVER, Los Angeles, CA (US)

Correspondence Address: MORRISON & FOERSTER LLP, 425 MARKET STREET, SAN FRANCISCO, CA, 94105-2482, US

(73) Assignee: INTRATHERAPIES LLC, Beverly Hills, CA (US)

(21) Appl. No.: 12/754,497

(22) Filed: Apr. 5, 2010

Related U.S. Application Data
(63) Continuation of application No. 11/701,288, filed on Jan. 31, 2007.
Application No. 11/701,288 is a continuation-in-part of application No. 10/382,213, filed on Mar. 5, 2003, now abandoned.
(60) Provisional application No. 60/416,316, filed on Oct. 4, 2002.

(51) Int. Cl. A61K 31/465; A61K 36/00; A61K 31/714; A61K 31/683; A61K 31/522; A61K 38/06; A61K 31/55; A61P 25/22; A61P 25/00; A61P 9/12; A61P 5/00; A61P 25/30; A61P 5/06; A61P 25/24; A61P 19/02; A61P 1/00; A61P 37/00; A61P 27/16

(52) U.S. Cl. 424/725; 514/283; 514/52; 514/77; 514/263.32; 514/18; 514/215

(57) ABSTRACT

Neuro-metabolic and endocrine-function regulating/modulating compositions, are disclosed. The compositions of the present invention comprise Selegiline Hydrochloride, Procaine Hydrochloride, Vinpocetine, trimethylglycine, and an ingredient selected from a group consisting of N-nicotinoyl-gamma-aminobutyric acid (N-GABA), niacin, niacinamide, gamma-aminobutyric acid (GABA), and combinations thereof. Methods of using the compositions, compositions, and compositions of the present invention are also disclosed.

### CROSS REFERENCE TO RELATED APPLICATIONS

This application is a Continuation of U.S. patent application Ser. No. 11/701,288 with a filing date of Jan. 31, 2007, which is a Continuation-in-part Application of U.S. patent application Ser. No. 10/382,213 with a filing date of Mar. 5, 2003, which claims priority to U.S. Provisional Patent Application Ser. No. 60/416,316 with a filing date of Oct. 4, 2002, all of which are incorporated herein by reference in their entirety.

### FIELD OF THE INVENTION

The invention relates generally to neuro-cerebral/neuro-metabolic and neuro-endocrine function regulating and modulating compositions. More particularly, the present invention relates to a neuro-degenerative inhibitor, neuro-endocrine modulator, and neuro-cerebral metabolism enhancer comprising Selegiline HCl, Procaine HCl, Vinpocetine, trimethylglycine, and an ingredient selected from the group consisting of N-nicotinyl-gamma-arninobutyric acid (N-GABA), niacin, niacinamide, gamma-aminobutyric acid (GABA), and combinations thereof.

### BACKGROUND OF THE INVENTION

In the recent decade, numerous medical studies have concluded that disorders or disturbances of the neuro-metabolic and neuro-endocrine function underlie a wide range of physical and mental impairments, including those associated with aging. It has been a major challenge in the medical field to study how deficits in brain-pituitary function and interruptions in the pathways conjoining the hypothalamus, adrenal and central/sympathetic nervous system arise with age. Studies are underway on the relationship of these deficits with neurological disorders, age-related decline of the striatal dopaminergic system, the degenerative process of glycation (pathological alterations causing proteins to react with sugars to form nonfunctioning structures), and progressive degeneration of systemic immunity, which leads to other diseases of internal organs and circulatory systems.

Within the body, there are a number of systems designed to maintain homeostasis, or normal human functioning. These systems work in concert helping the body to adapt to environmental changes and external stressors, both physically and mentally. Most stressful situations will induce the stimulation of a so-called Hypothalamo-Pituitary-Adreno-Sympathetic (HPAS) axis. The HPAS axis participates in the integration and management of information and stimulates physiological, biochemical, and endocrine responses that restore the physiological and biochemical equilibrium or homeostasis in the human body following a stressful event.

The metabolic events instigated by the HPAS axis may result, through a stimulation or an inhibition, in a change in the circulating level of catecholamines (noradrenaline and adrenaline) as well as other chemicals such as cortisol and aldosterone, each of which has metabolic effects on specific systems within the body, such as the cardiovascular, immune, neurological, and endocrine systems. For example, recent studies have suggested that dysfunction in serotonin receptor (5-hydroxytryptamine-1a; 5-HT-1a) activity observed in patients with depressive and anxiety disorders could be due to a hypersecretion of cortisol. (Neuropsychobiology 2001; 44(2):74-77.)

Accordingly, in humans, the maintenance of homeostasis under environmental stress lies largely under the control of the neuro-endocrine system. With age, there appears to be a decreased capacity to adapt to changes in the environment. Frequently, in older individuals, the response to environmental stress is delayed and of a lower magnitude.

A broad range of neuro-endocrine and neuro-metabolic medications and cognitive enhancers are available in the form of prescription medications. For example, there are a number of medications prescribed to improve mood and mental focus, mitigate depressive symptomology, and reduce anxiety, cravings, hyperactivity, and impulsivity. These medications include selective serotonin reuptake inhibitors (SSRI), such as those sold under the trademarks PROZAC® and PAXIL®, serotonin 2A antagonist and reuptake inhibitors (SARI), such as those sold under the trademarks SERZONE® and TRAZADONE HCI, serotonin and noradrenaline reuptake inhibitors (SNRI), such as SNRI sold under the trademark EFFEXOR® XR, noradrenaline and dopamine reuptake inhibitors (NDRI), such as NDRI sold under the trademark WELLBUTRIN SR®, dopamine reuptake inhibitors (DRI), such as DRI sold under the trademark RITALIN®, and serotonin and dopamine antagonist (SDA), such as SDA sold under the trademark RISPERDAL®. Additionally, neuro-endocrine medications and cognitive enhancers are available in a form of over-the-counter supplements, as well as alternative and homeopathic compositions. However, the available medications and compositions suffer from limited efficacy and significant side effects, such as insomnia, agitation, sexual dysfunction, fatigue, anxiety, adverse interaction with other supplements or prescription medications, and cannot efficiently prevent, control, and treat neural and endocrine dysfunctions.

### SUMMARY OF THE INVENTION

In view of the above-noted shortcomings of conventional neuro-endocrinal and neuro-metabolic medications and in view of the desire to develop the means for effective regulation and modulation of neuro-endocrinal and neuro-metabolic functions, it is an object of the present invention to provide a neuro-degenerative inhibitor, a neuro-endocrine modulator, and a neuro-cerebral metabolism enhancer that enable the mitigation of preexisting medical and/or age-related degenerative disorders with minimal toxicity and side effects. In particular, it is an object of the present invention to optimize formulation, dosage, and bioavailability of a chemical composition with neuro-endocrine modulating and neuro-cerebral/neuro-metabolic enhancing properties.

It is a further object of the present invention to provide a chemical composition with capabilities for the following:

(1) Increased catecholamine and neuronal stimulation and enhancement, optimizing catecholamine levels and their availability in the synapses for extended periods of time combined with optimized delivery of serotonin to the brain, in conjunction with improved efficiency of the neuronal firing rate within the ganglia;

(2) Regulation of the release of specific neurotransmitters by selective isolation and blockage of different classes of neuron-specific calcium channels;

(3) Mitigation or reversal of the age-related decline in neuronal receptor expression in the brain;

(4) Exerting antiaging and antidisease effects that are similar to those associated with caloric restriction (CR), which is known to effectively reduce the onset of age-related diseases and to retard the aging process in animals coupled with enhanced antioxidant enzyme induction;

(5) Mitigation of the age-related decrease in synaptic drive and loss of synapse associated with normal aging and further impacted by other diseases of the Central Nervous System (CNS);

(6) Enhancement of synaptic strength, modulation of neuronal signaling pathways, and associated hormonal functionality by enhancing rejuvenative and regenerative functionality of the hypothalamus, which is the part of the brain most intimately involved in the regulation of hormone secretion, pituitary glands, and endocrine glands, such as the adrenal glands, thyroid glands, testes, and ovaries. In particular, it is desired to provide a chemical composition with a capability to stimulate and enhance the composition ion of such hormones as luteinizing hormone (LH), follicle-stimulating hormone (FSH), adrenal corticotropic hormone (ACTH), thyroid-stimulating hormone (TSH), growth hormone (GH), and prolactin;

(7) Modulation and regulation of the magnocellular and parvocellular secretory regions of the hypothalamus, and enhancement of the input channels reaching the hypothalamus from a variety of sources, including other segments of the hypothalamus, diverse regions of the brain stem, and the forebrain;

(8) Optimization of hippocampal cell efficacy, particularly serotonin levels, to mediate or reverse cellular damage and mitigate depression- and anxiety-based disorders without the common side effects of conventional antidepression drugs, and mitigation of age-related changes in cellular proteins;

(9) Mitigation of sexual dysfunction side effects that are often associated with the use of SSRI-type antidepression medications. Approximately 60% of the estimated 150 million people receiving medication for depression are treated with SSRI-type drugs. It is currently estimated that 30%-40% of these patients, both male and female, experience some form of sexual dysfunction;

(10) Prophylaxis against nigrostriatal aging and secondary aging symptoms accompanying the decline of the dopaminergic nervous system and improvement of brain plasticity;

(11) Modulation of the molecular progression of aging and ongoing distortions in brain electrochemistry associated with age-related degenerative disorders; and

(12) Modulation of the inflammatory cascade leading to autoimmune disorders.

It is a further objective of the present invention to provide a chemical composition with capabilities for inhibiting the development of amyloidosis, a group of disabling diseases directly associated with aging and resulting from neurotoxic beta-amyloid deposits. It is particularly desirable to inhibit the formation of fibrils in amyloid proteins in order to treat amyloidosis.

It is a further object of the present invention to provide a chemical composition with capabilities for inhibiting the progressive degenerative effects of mitochondrial dysfunction by enhancing mitochondrial cellular energy and its cellular pathways. As free radicals are a by-composition of energy metabolism in the mitochondria, the composition targets the endogenous free radical production associated with the degenerative processes of aging.

It is still another object of the present invention to provide a chemical composition with capabilities for inhibiting primary aging pathologies, including neurofibrillary tangles, amyloid plaques, amyloid toxicity, inflammatory reactions, and microglial activations, thereby interrupting or retarding the age “triggers” associated with many CNS-related conditions.

It is also an object of the present invention to provide a chemical composition with capabilities for the facilitation of greater dynamic interaction and sensitization within the corpus callosum, the central region of human brain tissue that passes information and provides “messaging” functions between the two hemispheres of the brain as well as inhibiting the molecular mechanisms of neurodegeneration in the basal ganglia, leading to the dysfunction of neuronal pathways.

It is also an object of the present invention to provide a chemical composition with capabilities for enhancement of autoimmune responses and mitigation of anti-inflammatory conditions leading to bacterial and viral infections as well as immune system disorders. It is also an object of the present invention to provide a chemical composition with capabilities for the facilitation of enhanced microcapillary blood circulation within the skin.

These and other objects are achieved by providing a novel composition comprising Selegiline HC1, Procaine HCL, Vinpocetine, trimethylglycine, and an ingredient selected from a group consisting of N-nicotinyl-gamma-aminobutyric acid (N-GABA), niacin, niacinamide, gamma-aminobutyric acid (GABA), and combinations thereof. In one embodiment, the composition comprises 1 to 50 parts by weight of Selegiline Hydrochloride, 1 to 100 parts by weight of Procaine Hydrochloride, 1 to 50 parts by weight of Vinpocetine, 1 to 2,000 parts by weight of trimethylglycine, and 1 to 2,000 parts by weight of N-GABA ingredient selected from a group consisting of N-nicotinyl-gamma-aminobutyric acid (N-GABA), niacin, niacinamide, gamma-aminobutyric acid (GABA), and combinations thereof.

The composition may be in a form of a solid, a liquid, or an aerosol. The composition may further comprise therapeutic compounds, nutrition supplements, vitamins, herbs, homeopathic compositions, prescription medications, minerals, and trace elements. In one embodiment, the composition of the present invention further comprises a compound selected from a group consisting of acetyl-L-carnitine, methylcobalamin, glycerylphosphorylcholine, propentofylline, idebenone, pyritinol, piracetam, aniracetam, nefiracetam, oxiracetam, pramiracetam, levetiracelam, hydergine, modafinil, glutathione, centrophenoxine, and galantamine.

In one embodiment, optimal blood and brain uptake of the composition and its efficient migration through all neuro-cerebral pathways and neuro-endocrinc systems is further improved by administering the composition to patients in a form of sublingual infusion. It is an unexpected discovery of the present invention that sublingual infusion delivery appears to be more targeted than alternative methods, such as delivery through nasal membranes, injection, oral administration, and transdermal. The method of sublingual ingestion of the composition ensures enhanced bioavailability and transit through the blood brain barrier in the most direct and usable manner. For example, sublingual ingestion avoids dissipation and breakdown of ingredients, which is likely to occur when composition is orally ingested due to the intervention of gastric mucous and other interruptive actions of the digestive process.

In another aspect, the present invention provides a method of treating depression, sleep-pattern disorder, anxiety disorder, neurosis, neurasthenia, obsessive-compulsive disorder, chronic fatigue syndrome, digestive diseases, hypertension, osteoarthritis, and loss of libido. The method comprises providing the above-described composition and administering the composition to a patient in a single or multiple dosage regimen.

In another aspect, the present invention provides a method of reducing jet lag. The method comprises providing the above-described composition and administering the composition to a patient in a single or multiple dosage regimen.

In another aspect, the present invention provides a method of reducing inflammation. The method comprises providing the above-described composition and administering the composition to a patient in a single or multiple dosage regimen. In one embodiment, the inflammation is caused by arthritis, and the method is directed to the reduction of arthritic pain and swelling.

The present invention also provides a method of decreasing hormonal imbalance. The method comprises providing the above-described composition and administering the composition to a patient in a single or multiple dosage regimen.

The present invention also provides a method of autoimmune enhancement. The method comprises providing the above-described composition and administering the composition to a patient in a single or multiple dosage regimen.

The present invention also provides a method of combating vascular dementia and Alzheimer's disease. The method comprises providing the above-described composition and administering the composition to a patient in a single or multiple dosage regimen.

The present invention also provides a method of neuron and nerve cell regrowth. The method comprises providing the above-described composition and administering the composition to a patient in a single or multiple dosage regimen.

The present invention also provides a method of improving visual or auditory acuity. The method comprises providing the above-described composition and administering the composition to a patient in a single or multiple dosage regimen.

The above-mentioned features of this invention, and other, and the manner of obtaining them, will become more apparent, and will be best understood by reference to the following description.

### DETAILED DESCRIPTION OF THE INVENTION

Since serotonin levels are known to affect memory and emotions, an objective of the present invention is to provide a chemical composition that modulates and optimizes serotonin levels and creates antidepressant/antianxiety effects without the side effects of conventional drugs. Additionally, the chemical composition of the present invention has been formulated to provide a stimulating effect on the pituitary gland.

Also, it is known that acetylcholine (primarily from the nucleus of Meynert in the basal forebrain) acts as a neurotransmitter to stimulate the hippocampus, a biochemical basis of memory. Accordingly, the chemical composition of the present invention was formulated to facilitate acetylcholine production in order to improve memory function.

Many brain regions that are involved in regulating emotions and homeostasis have afferent and efferent connections with the hypothalamus. These regions include the amygdala, hippocampus, bed nucleus of the stria terminalis, dorsal raphe nucleus, locus coeruleus, parabrachial nucleus, and nucleus of the solitary tract. Neurons located in these brain regions can convey information relevant to the neuro-endocrine control to hypothalamic neurons and receive information from hypothalamic neurons. The processing and integration of the neuronal information flow relevant to the secretion of hormones is a primary focus of neuroendocrine research in antiaging, and was also central to the development of the chemical composition of the present invention.

In summary, the composition of the present invention was formulated to maintain and extend homeostasis and to treat a broad range of physical and mental impairments, including those associated with aging.

The composition of the present invention comprises a novel combination of Selegiline HCl, Procaine HCl Vinpocetine, trimethylglycine, and an ingredient selected from a group consisting of N-nicotinyl-gamma-aminobutyric acid (N-GABA), niacin, niacinamide, gamma-aminobutyric acid (GABA) and combinations thereof (this ingredient is referred to as “N-GABA ingredient” throughout the specification and claims). Although the active ingredients have a proven clinical history of long-term efficacy on an individual basis, as described below, they belong to different therapeutic classes and have not been integrated into a single composition prior to the present invention.

It is an unexpected discovery of this invention that when these ingredients are combined, they result in a composition with a number of synergistic beneficial effects in patients, including, but not limited to, the elimination of anxiety and hyperactivity; mitigation of panic symptomology; reduction of fatigue; improvement of self-confidence and self-esteem; elimination of the need for antidepressants; a cognitive enhancement, including improved concentration, clarity, and mental focus; improvement of aerobic endurance and performance and weight-training capacity; improvement of blood circulation; reduction of blood pressure and hypertension; improvement of sexual performance and libido; reduction of hormonal disbalance in menopausal and postmenopausal women; improvement of visual and auditory acuity; improvement of physical agility and muscle flexibility; improvement of social interaction skills; promotion of relaxation response and mitigation of chronic sleep-pattern disorders, such as insomnia and other chronic sleep disorders; reduction of arthritic pain and swelling, enhancement of immune system functioning; improvement of the metabolism, including relief from constipation; improvement of tone; mitigation of “jet lag”; and reduction in caffeine dependence.

The composition of the present invention may thus be used for the treatment of a broad spectrum of conditions and pathologies, including certain preexisting medical as well as age-related and neuro-degenerative conditions and pathologies. Some examples of diseases treatable with the instant composition are nerve diseases, including general anxiety disorder, depression, neurosis, neurasthenia, obsessive-compulsive disorder, chronic sleep disorder, and chronic fatigue syndrome, digestive diseases, including chronic constipation, hypertension, inflammatory diseases, including osteoarthritis and rheumatoid arthritis pain and swelling, and human sexual desire disorder.

A review of functionality profiles of patients treated with the composition of the present invention indicates some likely ongoing impact on the posterior lobe of the pituitary gland, also known as the neural lobe, because it is an extension of neurons originating in the hypothalamus. Also, it appears that the composition of the present invention has a catalyzing and potentiating effect on the intermediate lobe of the pituitary gland, which contains high concentrations of various peptides, including endorphin, melanocyte-stimulating hormone (MSH), and ACTH. Additionally, the composition appears to increase the levels of physiological markers, such as serotonin, dopamine, phenylethylalanine, superoxidase dismutase, catalase, nitric oxide, choline, and acetyl choline in the synapse and blood.

Additionally, the composition of the present invention appears to increase glucose and oxygen uptake at the cellular level from the blood. Also, the composition appears to block the effects of Corticotropin Releasing Factor (CRF). CRF is known to stimulate hypersecretion of cortisol in patients suffering from depression (Corticotropin-Releasing Factor Triggers Depression; Clinical Psychiatry News, 6/00). The composition is also believed to reduce levels of free radicals by forming antioxidants and to enhance blood circulation, including accelerated blood flow to facial microcapillaries, which may provide benefits to the dermis (cortium) and epidermis.

In one embodiment, the chemical composition of the present invention contains the active ingredients in the following ranges (in mg per day):

| | |
|---|---|
| Selegiline Hydrochloride | 1-50 |
| Procaine Hydrochloride | 1-100 |
| Vinpocetine | 1-50 |
| N-GABA ingredient | 1-2000 |
| Trimethylglycine | 1-2000 |

Preferably, the chemical composition of the present invention contains the active ingredients in the following ranges (in mg per day):

| | |
|---|---|
| Selegiline Hydrochloride | 1-10 |
| Procaine Hydrochloride | 25-75 |
| Vinpocetine | 1-10 |
| N-GABA ingredient | 1-100 |
| Trimethylglycine | 300-700 |

In one embodiment, the chemical composition is formulated as follows (mg per day):

| | |
|---|---|
| Selegiline Hydrochloride | 2.5 |
| Procaine Hydrochloride | 50 |
| Vinpocetine | 5 |
| N-GABA ingredient | 50 |
| Trimethylglycine | 500 |

It should be apparent to those skilled in the art that the composition of the present invention may be formulated as a single dose for administration to the patient once a day or as a number of doses that add up to the amount specified above.

A. Selegiline Hydrochloride

Selegiline Hydrochloride (Selegiline) is also known as L-deprynyl, Jumex®, ELDEPRYL™, MOVERGAN™, and (R)-(−)-N,2-dimethyl-N-2-propynylphenethylamine. Selegiline is best known as an irreversible inhibitor of monoamine oxidase-B (MAO-B), whose increased activity has been associated with the aging process. The MAO-B enzyme also constitutes the main degrading pathway for dopamine, the transmitter that is deficient in the brain of Parkinson's disease patients. In each decade over age 45, humans lose about 13% of dopamine-producing neurons. In Parkinson's disease, close to 80% of these neurons are dead. Currently, there is no long-term therapy to reverse Parkinson's disease. However, it is believed that by controlling MAO levels, much of the damage to brain cells that aging causes may be avoided, thereby preventing and somewhat mitigating diseases like Parkinson's and other forms of senile dementia.

Selegiline Hydrochloride is primarily used for administration to Parkinsonian patients receiving levodopa/carbidopa therapy and who demonstrate a deteriorating response to this treatment. The recommended regimen for the administration of Selegiline Hydrochloride is 10 mg per day, administered as divided doses of 5 mg each and taken at breakfast and lunch. Higher doses are ordinarily avoided because of the increased risk of side effects.

Additionally, it has been reported that 10 mg of Selegiline HCL per day may improve attention and episodic memory in Parkinson's disease and early Alzheimer's disease patients. Selegiline also improves motor reactions in Parkinson's patients and subjective feelings of increased vitality, euphoria, and energy. At doses of between 10 and 40 mg a day, it has also been shown to improve depression, particularly when psychomotor retardation is prominent and anxiety minimal (Lees A. J., Deprenyl and Cognition, Acta Neurol Scand Suppl, 1991; 136:91-4).

Selegiline has also been shown to protect nerve cells against a wide number of neurotoxins (Maroyama, W. et al. (1998), (−)-Deprenyl protects human dopaminergic neuroblastma SH-SY5Y cells from apoptosis induced by peroxynitrite and nitric oxide, J Neurochem, 1996; 70:2510-15; Magyar, K. et al., The pharmacology of B-type selective monoamine oxidase inhibitors; milestones in (−)-deprenyl research, J Neural Transm Suppl, 48:29-43). Selegiline is also shown to work as a neuroprotection composition when nerve cells are exposed to damaging or stressful conditions (Tatton, W. G. et al. (1996), (−)-Deprenyl reduces neuronal apoptosis and facilitates neuronal outgrowth by altering protein synthesis without inhibiting monoamine oxidase, J Neural Transm Suppl, 48:45-59). In aged rats, deprenyl has proven to be a highly effective “sexual rejuvenator.” (Knoll, J. (1997), Sexual performance and longevity, Exp Gerontal, 32: 539-52.) Selegiline has also proven in four different rat studies and one dog study to be an effective life-extension composition (Knoll, J. (1995), Rationale for (−)-deprenyl (selegiline) medication in Parkinson's disease and in prevention of age-related nigral changes, Biomed Pharmacothe, 1997; 49:187-95; Ruehl, W. et al., Treatment with L-deprenyl prolongs life in elderly dogs, Life Sci 61, 1037-44).

Other positive effects of Selegiline reported by various research groups include a possible delay of the deterioration of neurons during aging (Maruyama, W. and Naoi, M., Neuroprotection by deprenyl and related compounds, Mech Ageing Dev, 1999 November; 111(2-3):189-200); a decrease of susceptibility to Parkinson's & Alzheimer's disease (Knoll J., Deprenyl (Selegiline): past, present and future, Neurobiology (Bp), 2000; 8(2):179-99); protection of the vascular endothelium from beta amyloid plaque (Thomas, T. et al., L-deprenyl: nitric oxide production and dilation of cerebral blood vessels; Neuroreport, 1998 Aug. 3; 9(11):2595-600); a reduction of cocaine “high” and cocaine dependence (Newton, T. F. et al., Effects of selegiline pretreatment on response to experimental cocaine administration, Psychiatry Res, 1999 Oct. 11; 87(2-3):101-6; Bartzokis, G. et al., Selegiline effects on cocaine-induced changes in medial temporal lobe metabolism and subjective ratings of euphoria, Neuropsychopharmacology, 1999 June; 20(6):582-90); a reduction of oxidative damage to the brain (Kitani, K., Assessing the effects of deprenyl on longevity and antioxidant defenses in different animal models, Ann NY Acad Sci, 1998 Nov. 20; 854:291-306); a reduction of oxidative stress and an increase of free radical elimination (J. Knoll, A review of the pharmacology of selegiline, Acta Neurol Scand Suppl Denmark, 1991; 84(136):44-59); an inhibition of tumor growth in rats with mammary tumors; a protection of cells from the DNA damage (ThyagaRajan, S. and Quadri, S. K., Deprenyl inhibits tumor growth, reduces serum prolactin, and suppresses brain monoamine metabolism in rats with carcinogen-induced mammary tumors, Endocrine, 1999 June; 10(3):225-32); and a stimulation of biosynthesis of cytokines interleukin-1 & 6, elevated levels of which are usually associated with immune response to bacterial or viral infection (Wilfried, K. et al., Selegiline stimulates biosynthesis of cytokines interleukin-1 beta and interleukin-6, Neuroreport, 1996 Nov. 25; 7(18):2847-8).

Accordingly, some researchers recommend that healthy adults take orally 1.5-2 mg/day of Selegiline, starting around age 40, possibly even in the 30s, in order to combat neuron death associated with aging (James South, M. A., Deprenyl—extending lifespan).

Although certain undesirable side effects, such as irritability, hyperexcitability, psychomotor agitation, insomnia, elevated blood pressure levels, as well as sporadic cases of neck stiffness, are known to be associated with the use of Selegiline, currently, there is no formulation containing Selegiline that effectively overcomes these side effects while preserving the beneficial effects of Selegiline. For example, some researchers recommend the use of calming/sleep-inducing serotonergic systems, such as magnesium and tryptophan, or 5-HTP (James South, M. A., Deprenyl—extending lifespan). However, 5-HTP and tryptophan are known depressants or semisedative hypnotic compounds. Additionally, in one study, a combination of L-deprenyl (5-10 mg/day) with L-phenylalanine (205 mg/day) was shown to have high antidepressive efficacy (Birkmayer, W., L-deprenyl plus L-phenylalanine in the treatment of depression, J Neural Transm, 1984; 59(1):81-87).

However, none of the references cited above suggest combining Selegiline with the other ingredients of the instant composition. It is unexpected discovery of the present invention that the composition enables one to achieve the beneficial effects of Selegiline while avoiding its undesirable side effects. This result is achieved without introducing additional negative side effects that are commonly associated with other additives, such as 5-HTP and tryptophan, used in a combination with Selegiline.

As will be discussed in more detail in the Examples Section that follows, the composition of the present invention overcomes side effects associated with an individual use of Selegiline. The patients treated with the composition of the present invention did not complain about irritability, hyperexcitability, psychomotor agitation, insomnia, and elevated blood pressure levels. To the contrary, they reported an elimination of anxiety symptoms (Example 5), an elimination of symptoms of Attention Deficit Hyperactive Disorder (ADHD) (Example 8), an improved alertness and ability to focus (Examples 3, 4, 6, and 8), a restful sleep (Examples 2, 3, and 5), a reduction and stabilization of blood pressure (Example 3), and an improvement of physical endurance and agility (Examples 5 and 7). Similarly, the patients treated with the composition of the present invention were free from depression, a side effect associated with the use of magnesium and tryptophan, or 5-HTP in a combination with Selegiline. To the contrary, the composition was found to be effective in treating depression (Examples 5 and 8).

B. Procaine Hydrochloride

Procaine HCL (Procaine) is also known as novocain hydrochloride and 2-diethylaminoethyl p-aminobenzoate hydrochloride. Procaine is a short-acting, low potency, local anesthetic of the ester type used for local, regional, and short-acting spinal anesthesia. Anesthesia is obtained within 2-5 minutes and lasts approximately 1 hour.

Once absorbed, the procaine hydrochloride salt is rapidly hydrolyzed by plasma cholinesterase into p-amino benzoic acid (PABA) and diethylaminoethanol (DEAE). PABA is a well-known vitamin that is often considered necessary for the growth of poultry, for the growth of healthy intestinal flora, and for the maintenance of a normal fur coat in fur-producing animals. It has also been suggested as a vitamin important to skin and hair pigmentation. DEAE is also biologically active. It is closely related to its adjacent homolog dimethylaminoethanol (DMAE), which is precursor of the vitamin choline. DEAE also participates in the synthesis of choline and acetylcholine. DEAE has also been reported to produce mental stimulation, mild euphoria and, unlike other mental stimulants such as amphetamines, has no adverse side effects such as depression. (U.S. Pat. No. 5,254,686.)

In the late 1950s, a composition marketed as GH-3, consisting of procaine hydrochloride (active ingredient), ascorbic acid, citric acid, benzoic acid, potassium metabisulphite, and disodium phosphate, was suggested for a number of therapeutic uses. It has been suggested that GH-3 may be used as an antidepressant, a reversible and competitive monoamine oxidase (MAO) inhibitor. GH-3 has also been shown to be highly effective in treating paroxysmal supraventricular tachycardia, anterial fibrillation, and complete heart block. GH-3 was also claimed to improve skin conditions, sleep patterns, blood pressure, and heart arrhythmia, to reduce hair loss and graying, and the wrinkling and hardening of skin (Conrad S Myers, GH3: Procaine Hydrochloride, Positive Health). The typical recommended dosage of GH-3 was 100 mg to 200 mg daily.

Another procaine-based composition, KH3, comprises a combination of procaine with hematoporphyrin, which is believed to enhance the activity of procaine. It has been reported that KH3 may reduce urinary incontinence in the elderly (Procaine haematoporphyrin (K.H.3) and its effect on urinary incontinence in the institutionalized elderly. J Clin Exp Gerontol (USA), 1987; 9(1):43-55); may work as an antidepressant (A procaine derivative for the treatment of depression in an outpatient population, Psychosomatics (USA), 1976; 17(2):96-102); and may have antioxidant and lipid-lowering effects (Antioxidant and lipid-lowering effects of the original procaine-based compositions, Romanian J of Gerontol and Geriat (Romania), 1996; 18(3-4):47-61).

In the late 1990s, Steroidogenesis Inhibitors (STGI) and Altachem Pharma began clinical testing of ANTICORT™, a still another composition containing procaine hydrochloride (HCI), as an anticortisol medication in AIDS patients. It is generally believed that very high levels of cortisol, a stress-associated hormone, may have an immunosuppressive effect, may lead to a sexual dysfunction and infertility, adrenal hyperplasia, thinning of the skin, hypertension, psychological disturbances, abnormal fat deposition (J Clin Investi, October 2001, 108(8), 8, 1123-1131), loss of muscle and bone mass, cognitive impairment, and dementia.

ANTICORT™ is being evaluated in HIV positive patients under anti-HIV therapy, to determine its potential effect on various immune systems.

The most notable side effects reported by patients receiving GH3 and KH3 were heartburn and bloating. Additionally, a number of studies have described other side effects associated with the use of procaine. For example, loss of vision following injection of procaine for extraction of teeth has been reported. (Grant, W. M., Toxicology of the Eye. 3rd ed. Springfield, Ill.: Charles C. Thomas Publisher, 1986.) It has been also reported that use of procaine as an analgesic may lead to such side effects as tachycardia, dyspnea, anxiety, and disorientation. (Goodman, L. S., and A. Gilman. (eds.), The Pharmacological Basis of Therapeutics. 5th ed. New York: Macmillan Publishing Co., Inc., 1975.)

Furthermore, when taken in large doses procaine was reported to produce restlessness and tremors proceeding to convulsions, followed by depression and death due to respiratory failure. However, in some cases, even small doses, of procaine resulted in cardiovascular collapse and death. (Gosselin, R. E., R. P. Smith, H. C. Hodge. Clinical Toxicology of Commercial Compositions. 5th ed. Baltimore: Williams and Wilkins, 1984.) Clearly, there is no consensus in the medical research community on beneficial and negative properties of procaine. While some studies report that use of procaine may result in certain heart and respiratory problems, others indicate that procaine-containing compositions GH-3 and K_H3 are highly effective in treating heart conditions.

The composition of the present invention appears to overcome a broad range of side effects of procaine. Clinical observations of patients treated with the composition of the present invention indicate its cardioprotective properties. High blood pressure is one of the key statistical markers for heart disease and stroke. The composition of the present invention lowers blood pressure (Example 3) and thus reduces a probability of stroke. The composition also appears to improve aerobic endurance (Examples 5 and 7), which may indicate an improvement in overall cardiovascular health. Additionally, patients using the composition of the present invention reported improvement in visual acuity (Example 6), an elimination of anxiety symptoms (Example 5), an elimination of symptoms of hyperactivity (Example 8), and an improved alertness, clarity, and ability to focus (Examples 3, 4, 6, and 8). Also, unlike the users of GH-3 and KH3, the patients using the composition of the present invention have reported no heartburn and bloating. To the contrary, they have indicated a more effective digestive process and elimination of bloating or fullness around the abdomen.

Furthermore, prior to the present invention, additional benefits obtained by combining procaine with the other active ingredients of the instant chemical composition have not been realized by those skilled in the art. In fact, it was a general belief that concurrent use of monoamine oxidase (MAO) inhibitors, such as Selegiline, in patients receiving local anesthetics, such as procaine, may increase the risk of hypotension. (Drug Information for the Health Care Professional. (19th ed.), Vol. I. Micromedex, Inc. Englewood, Colo., 1999.) Hypotension is a sudden fall in blood pressure that occurs when a person assumes a standing position. Symptoms, which generally occur after sudden standing, include dizziness, lightheadedness, blurred vision, and syncope (temporary loss of consciousness). (National Institute of Neurological Disorders and Stroke Web page.) The composition of the present invention, which combines Selegiline and procaine with Vinpocetine, trimethylglycine, and N-GABA ingredient, unexpectedly stabilizes blood pressure at a normal level (Example 3), reduces fatigue and increases energy level (Examples 4, 5, and 6), improves alertness, clarity, and ability to focus (Examples 3, 4, 6, and 8), and improves visual acuity (Example 6).

C. Vinpocetine

Vinpocetine (also referred to as Apovincamine, Vincamine, Cavinton, Vinca minor, and ethyl apovincaminate) is a periwinkle plant extract (Vinca major) that is believed to improve brain function. It has been used medicinally in Europe since 1983.

Vinpocetine has been shown to be a cerebral metabolic enhancer and a selective cerebral vasodilator (B. Vamosi et al. (1976), Comparative study of the effect of Ethyl Apovincaminate and Xanthinol Nicotinate in cerebrovascular diseases, Arzneim Forsch (drug research) 28, 1980-84; F. Solti et al. (1976), Effect of Ethyl Apovincaminate on the cerebral circulation, Arzneim Forsch (drug research) 28, 1945-47; James South, M. A., Vinpocetine—a cerebral metabolic enhancer). Also, Vinpocetine has been shown to enhance oxygen and glucose uptake from the blood by brain neurons, to increase neuronal ATP bioenergy production, and to increase the tolerance of the brain toward hypoxia and ischemia (E. Karpaty and L. Szporny (1976), General and cerebral harmodynamic activity of Ethyl Apovincaminate, Arzneim Forsch (drug research) 28, 1908-12; A. Szobor and M. Klein (1976), Ethyl Apovincaminate therapy in neurovascular disease, Arzneim Forsch (drug research) 28, 1984-89; James South, M. A., Vinpocetine—A cerebral metabolic enhancer).

Additionally, Vinpocetine has been shown to significantly improve chronic cerebral dysfunction in elderly patients (A double-blind placebo controlled evaluation of the safety and efficacy of Vinpocetine in the treatment of patients with chronic vascular senile cerebral dysfunction, Am Geriatr Soc, May 1987; 35(5):425-30); enhance the brain circulation and oxygen use without the significant alteration in parameters of systemic circulation, provide an anticonvulsant activity, inhibit the effect on phosphodiesterase (PDE) enzyme and improve rheological properties of the blood (On the mechanism of action of vinpocetine, Acta Pharmaceutica Hungarica (Hungary), 1996, 66(5):213-224). Evidence has been obtained that the neuroprotective action of Vinpocetine is related to the inhibition of the operation of voltage-dependent neuronal Na⁺-channels, indirect inhibition of some molecular cascades initiated by the rise of intracellular Ca²⁺-levels, and, to a lesser extent, inhibition of adenosine reuptake (On the mechanism of action of vinpocetine, Acta Pharmaceutica Hungarica (Hungary); 1996, 66(5):213-224). Typically, it is recommended to take 5-10 mg of Vinpocetine two to three times each day (James South, M. A., Vinpocetine—A cerebral metabolic enhancer).

However, prior to the present invention, additional benefits obtained by combining Vinpocetine with the other active ingredients of the instant chemical composition have not been realized by those skilled in the art. It is believed that a neurophysiological activity of Vinpocetine increases at least tenfold when it is incorporated into the composition of the present invention as compared to its individual use. It is also believed that other ingredients of the composition of the present invention work synergistically with Vinpocetine to enhance its calcium-blocking effects. This results in a greater blood flow to the brain and other organs (Example 4), an increase in the body's ability to generate energy (Examples 4, 5, and 6), an improvement of physical endurance and agility (Examples 5 and 7), improvement of sexual performance and libido (Examples 5 and 6), and improved visual acuity (Example 6).

Additionally, the present composition provides a number of positive effects that are not achievable by the use of Vinpocetine on its own. These positive effects include elimination of anxiety and hyperactivity; mitigation of panic symptomology; improvement of self-confidence and self-esteem; elimination of the need for antidepressants; improvement of aerobic endurance and performance and weight-training capacity; improvement of social interaction skills, mitigation of chronic sleep-pattern disorders, reduction of arthritic pain and swelling, enhancement of immune system functioning, improvement of the metabolism, mitigation of “jet lag,” and reduction in caffeine dependence, to name a few.0

D. N-GABA Ingredient

N-GABA ingredient of the present invention is an ingredient selected from a group consisting of N-nicotinyl-gamma-aminobutyric acid (N-GABA), niacin, niacinamide, gamma-aminobutyric acid (GABA), and combinations thereof.

N-GABA is also referred to as PICAMILON™, Picamilion, Pycamilon, and sodium salt of N-nicotinyl-gamma-aminobutyric acid or sodium nicotinyl-aminobutyrate. N-GABA is niacin (vitamin B3) bonded to gamma amino butyric acid. The normal dosage is 100 to 500 mg.

N-GABA has been shown to induce a significant increase of cerebral blood flow (Mirzoian, R. S. and Gan'shina, T. S., N-GABA Enhances Blood Flow; Farmakol Toksikol (USSR) January-February 1989; 52(1):23-6) and benefit in the treatment of pigmented retinal abiotrophy (PRA) (Davydova, G. A. et al., Vasoactive composition Picamilion in pigmented retinal abiotrophy, Vestnik Oftalmologii (Russian Federation), 1995; 111(3):20-22). It is generally believed that when niacin is bound to GABA, it creates a molecule that readily penetrates the blood-brain barrier to enhance cerebral and peripheral circulation.

Additionally, based on a clinical investigation in 16 neurologic and psychiatric clinics on 984 patients, N-GABA was recommended by the Pharmacological Committee of the Ministry of Health of the former USSR for application in medicine for stroke therapy, for the treatment of transient disorders and chronic insufficiency of blood circulation, and also as a tranquilizer without the sedative component and myorelaxation; and for the treatment of asthenic disorders and depressions of old age. As a therapeutic and prophylactic remedy, N-GABA was recommended for increasing the stability toward physical and psychological burden (A. P. Huaichenko and R. P. Kruglikova-Lvova, Picamilon, The Pharmacological Committee of the Ministry of Health of USSR). N-GABA is also believed to have a mild tranquilizing effect without sedative action, and to reduce symptoms of anxiety and depression.

Although picamilone is considered to be generally safe, it has been shown to produce certain side effects, including headaches, dizziness, nausea, and insomnia (particularly when taken in late evening). The composition of the present invention appears to overcome these side effects of picamilone. Not only do the patients treated with the composition of the present invention not report these side effects, they also reported an improved alertness, clarity, and ability to focus (Examples 3, 4, 6, and 8), a restful sleep (Examples 2, 3, and 5), improved aerobic endurance (Examples 5 and 7), and an increase in energy (Examples 4, 5, and 6).

Furthermore, prior to the present invention, additional benefits obtained by combining N-GABA ingredient with the other active ingredients of the instant chemical composition have not been realized by those skilled in the art. The present composition provides a number of positive effects that are not achievable by the use of N-GABA ingredient on its own. These positive effects include improved concentration, clarity, and mental focus, elimination of hyperactivity, improvement of muscle flexibility, improvement of social interaction skills, reduction of arthritic pain and swelling, reduction of hormonal disbalance, enhancement of immune system functioning, improvement of the metabolism, mitigation of “jet lag,” and reduction in caffeine dependence, to name a few.

E. Trimethylglycine

Trimethylglycine is also referred to as Betaine. Trimethylglycine is a methyl donor, and as such, has been suggested to be important for proper liver function, cellular replication, and detoxification reactions. Trimethylglycine also plays a role in the production of carnitine and serves to protect the kidneys from damage (Chambers, S. T., Betaines: Their significance for bacteria and the renal tract. Clin Sci, 1995; 88:25-7). Trimethylglycine is primarily used as a nutritional supplement in supporting proper liver function. The typical recommended dose for trimethylglycine citrate or trimethylglycine aspartate is 1,000 to 2,000 mg three times a day.

Additionally, trimethylglycine has been-reported to have anticonvulsant properties (Freed, W. J., Prevention of strychnine-induced seizures and death by the N-methylated glycine derivatives betaine, dimethylglycine and sarcosine, Pharmacol Biochem Behav, 1985; 22(4):641-3) and the ability to improve lipid metabolism (Panteleimonova T N; Zapadniuk V I, Effect of trimethylglycine on lipid metabolism in experimental atherosclerosis in rabbits, Farmakol Toksiko, (USSR), July-August 1983; 46(4):83-5). However, prior to the present invention, additional benefits obtained by combining trimethylglycine with the other active ingredients of the instant chemical composition have not been realized by those skilled in the art.

Trimethylglycine, a methyl donor compound, would appear to be an unlikely addition to the chemical blend of neuro-cerebral/neuro-endocrine/anti-inflammatory compounds because it is not categorized as a nootropic. Trimethylglycine is primarily viewed as having general cardioprotective features, essentially due to its effectiveness in reducing homocysteine levels, a toxic breakdown composition of amino acid metabolism that is believed to promote atherosclerosis and osteoporosis, and supporting proper liver function.

The inclusion of trimethylglycine in the composition of the present invention is predicated upon the belief that it will carry and donate methyl molecules to facilitate an enlarged range of necessary chemical interactions. It is also believed that the ability of the body to use the methyl from trimethylglycine is limited when trimethylglycine is taken alone by the patient. On the other hand, when trimethylglycine is incorporated into the composition of the present invention, it acts synergistically with other active ingredients, resulting in a number of beneficial neurophysiological effects that cannot be achieved by using trimethylglycine on its own. These positive effects include the elimination of anxiety and hyperactivity; mitigation of panic symptomology; reduction of fatigue; improvement of self-confidence and self-esteem; elimination of the need for antidepressants; a cognitive enhancement, including improved concentration, clarity, and mental focus; improvement of aerobic endurance and performance and weight-training capacity; improvement of sexual performance and libido; reduction of hormonal disbalance; improvement of visual and auditory acuity; improvement of physical agility and muscle flexibility; improvement of social interaction skills; promotion of relaxation response and mitigation of chronic sleep pattern disorders; mitigation of “jet lag”; and reduction in caffeine dependence, to name a few.

While not wanting to be bound by theory, it is believed that the synergistic effect of the instant composition is based on the interaction of Selegiline, Procaine, Vinpocetine, and N-GABA, further enhanced by the infusion of the concentrated methyl donor molecules obtained from the addition of a fifth compound, Trimethylglycine (TMG). The corollary anti-inflammatory effects, as evident in the beneficial impact on pain and swelling associated with osteoarthritis and rheumatoid arthritis, and the cardiovascular benefits of the composition may result from the conversion of homocysteine into methionine by TMG, which is further converted into S-adenosyl-methionine (SAMe). This sequence is believed to provide favorable results while avoiding side effects commonly associated with conventional steroidal and nonsteroidal anti-inflammatory drugs. Formation of SAMe may further facilitate antidepressant effectiveness of the composition as a result of the chemical interaction with the other four ingredients.

It is further believed that SAMe may participate in the sequential series of actions that increase glutathione levels in the brain cells, inhibit lipid peroxidation, and stimulate enzymatic actions throughout the body. Such enzyme-enhancing action, combined with the activity of the other four ingredients, may be responsible for the high efficiency of the composition in alleviating depression.

In one embodiment, the composition comprises such amounts of each ingredient that, when the composition is administered to a patient, at least one health condition is relieved, wherein the health condition is selected from a group consisting of anxiety, hyperactivity, panic symptoms, fatigue, hypertension, hormonal disbalance, jet lag, substance dependence, insomnia, loss of appetite, and emotional disturbance. In one embodiment, the substance dependence is a caffeine dependence. In another embodiment, the hormonal disbalance is a hormonal disbalance in menopausal and postmenopausal women.

In another embodiment, the composition comprises such amounts of each ingredient that, when the composition is administered to a patient, a concentration of at least one chemical species is increased, wherein the chemical species is selected from the group consisting of serotonin, choline, acetylcholine, endorphin, melanocyte-stimulating hormone (MSH), ACTH, dopamine, phenylethylalanine, superoxidase dismutase, catalase, antioxidants, and nitric oxide. In still another embodiment, the composition comprises such amounts of each ingredient that, when the composition is administered to a patient, levels of at least one chemical selected from a group consisting of cortisol and free radicals is reduced.

In another embodiment, the composition comprises such amounts of each ingredient that, when the composition is administered to a patient, a function of the pituitary gland is stimulated. In another embodiment, the composition comprises such amounts of each ingredient of the composition that, when the composition is administered to a patient, a cellular uptake of oxygen and glucose is increased as compared to the pretreatment levels.

In another embodiment, the composition comprises such amounts of each ingredient of the composition that, when the composition is administered to a patient, symptoms of at least one disease or disorder selected from a group consisting of depression, sleep pattern disorder, anxiety disorder, neurosis, neurasthenia, obsessive-compulsive disorder, chronic fatigue syndrome, digestive diseases, hypertension, osteoarthritis, and loss of libido, is mitigated.

In another embodiment, the composition comprises such amounts of each ingredient of the composition that, when the composition is administered to a patient, at least one neuro-endocrine or physiological improvement is observed or reported by the patient. Neuro-endocrine or physiological improvement may include increased self-confidence, increased self-esteem, elimination of the need for anti-depressants, cognitive enhancement, improvement of aerobic endurance, improvement of weight-training capacity, improvement of blood circulation, reduction of blood pressure, improvement of sexual performance, improvement of libido, improvement of visual acuity, improvement of auditory acuity, improvement of physical agility, improvement of muscle flexibility, improvement of social interaction skills, promotion of relaxation response, reduction of arthritic pain and swelling, enhancement of immune system functioning, improvement of metabolism, and improvement of skin tone. In one embodiment, the improvement of metabolism is a relief from constipation. In another embodiment, the cognitive enhancement comprises improvement of a mental ability selected from a group consisting of concentration, clarity, alertness, and mental focus.

As discussed above, the composition of the present invention unexpectedly overcomes undesirable side effects associated with individual use of each ingredient of the composition. Accordingly, in one embodiment, the composition comprises sufficient amounts of Selegiline Hydrochloride, Procaine Hydrochloride, Vinpocetine, N-GABA ingredient, and trimethylglycine to compensate for undesirable side effects associated with the administration of each ingredient on its own. The undesirable side effects may include irritability, hyperexcitability, psychomotor agitation, insomnia, elevated blood pressure levels, heartburn, bloating, dizziness, nausea, headaches, blurred vision, disorientation, tachycardia, and dyspnea.

The composition of the present invention may be delivered to a patient by a number of means, including sublingual infusion, delivery through nasal membranes, injection, oral administration, and transdermal delivery. However, it is believed that the sublingual infusion is more targeted, convenient, and effective as compared to the alternative delivery methods. For example, delivery through nasal membranes may be irritating to the nasal mucosa and may be inefficient, because it requires a dissolution of the composition prior to the administration. Injection is inconvenient for self-administration. Oral administration may limit the efficacy of the composition due to a gastric acid breakdown of the composition and filtering out of the composition by the liver before the composition reaches the blood-brain barrier. Also, transdermal delivery may be less efficient than sublingual infusion because it requires the diffusion of the composition through the dermal layers.

Accordingly, in one embodiment of the present invention, the composition is administered to the patients by sublingual infusion. The delivery mechanism of sublingual infusion is believed to enable the composition to cross the blood-brain barrier and enter the central nervous system (CNS) without the threat of breakdown in the gastric acid or on the initial passage through the liver. It is also believed that the optimal dosing and bioavailable delivery attributable to sublingual ingestion are important factors in the stability and neuro-systemic efficacy of the composition of the present invention.

The composition of the present invention may be in a form of a solid, a liquid, or an aerosol. For example, the composition may be in a form of an oral tablet, a capsule, a powder, an aerosol, a nebulized vapor, or a transdermal patch. In one embodiment the composition is suitable for parenteral or oral administration. The parenteral administration may be selected from a group consisting of intradermal, subcutaneous, intramuscular, intravenous, intrathecal, sublingual, rectal, vaginal, intraocular, transdermal, respiratory, mucosal, and pulmonary routes of administration. In one embodiment, the solid composition of the present invention is a finely granulated powder for sublingual administration. In another embodiment, the composition is suitable for a sublingual administration.

A composition of the present invention may further include one or more formulation additives used in food and drug compositions. Such formulation additives are well-known to those skilled in the art and may be selected from the group consisting of water, alcohols, amylaceous substances, thickeners, such as gums, fibers, lipids, fatty acids, including essential fatty acids, vitamins, including ascorbic acid and glycosyl ascorbic acids, minerals, including salts of magnesium and calcium, flavors, coloring compositions, sweeteners, seasonings, separators, preservatives, carriers, excipients, adjuvants, diluents, and stabilizers, to name a few. For example, in one embodiment, acesulfame potassium is used as a sweetener. In another embodiment, AVICEL™ (microcrystalline α-cellulose) is used as a separator that prevents the clumping of the ingredients and insures better sublingual adsorption of the composition.

The composition of this invention may include other ingredients, such as therapeutic compounds, nutrition supplements, vitamins, herbs, homeopathic compositions, other prescription medications, minerals, and trace elements. In one embodiment, the composition of the present invention further comprises at least one compound selected from the group consisting of hematoporphyrin, acetyl-L-carnitine, methylcobalamin, glycerylphosphorylcholine, propentofylline, idebenone, pyritinol, piracetam, aniracetam, nefiracetam, oxiracetam, pramiracetam, levetiracetam, hydergine, glutathione, modafinil, centrophenoxine, and galantamine.

For example, in one embodiment, an additional ingredient is hematoporphyrin. Hematoporphyrin is believed to boost the action of procaine, increasing its bioavailability by a factor of three. An amount of hematoporphyrin added to the composition of the present invention may be in the range from 1 mg to 2 g.

In another embodiment, an additional ingredient is acetyl-L-carnitine. Acetyl-L-carnitine has been reported to have beneficial effects on both clinical and CNS neurochemical parameters in Alzheimer's disease (Pettegrew, J. W. et al., Clinical and neurochemical effects of acetyl-L-carnitine in Alzheimer's disease, Neurobiology of Aging, January-February 1995; 16(1):1-4) and sleep patterns (Clinical pharmacodynamics of acetyl-L-carnitine in patients with Parkinson's disease, Int J Clin Pharmacol Res, 1990; 10(1-2):139-43). Acetyl-L-carnitine has been shown to facilitate the repair of transacted sciatic nerves (Acetyl-L-carnitine corrects the altered peripheral nerve function of experimental diabetes, Metabolism: Clinical and Experimental (USA), 1995; 44(5): 677-680.) Acetyl-L-carnitine also appears to reverse many age-associated deficits in cellular function, in part by increasing cellular ATP production (Oxidative damage and mitochondrial decay in aging; Proc Natl Acad Sci; Nov. 8, 1994). An amount of Acetyl-L-Carnitine added to the composition of the present invention may be in the range from 1 mg to 2 g.

In another embodiment, an additional active ingredient is methylcobalamin. Methylcobalamin is a coenzyme form of vitamin B12. Studies indicate that methylcobalamin increases the recovery time for facial nerve function in Bell's palsy (Jalaludin, M A. Methylcobalamin treatment of Bell's palsy. Methods Find Exp Clin Pharmacol, 1995; 17:539-544); inhibits the proliferation of malignant cells (Nishizawa, Y., Yamamoto, T., Terada, N., et al. Effects of methylcobalamin on the proliferation of androgen-sensitive or estrogen-sensitive malignant cells in culture and in vivo. Int J Vitam Nutr Res, 1997; 67:164-170); alleviates diabetic neuropathy (Yaqub, B. A., Siddique, A., Sulimani, R. Effects of methylcobalamin on diabetic neuropathy. Clin Neurol Neurosurg, 1992; 94:105-111); improves eye function (Kikuchi, M., Kashii, S., Honda, Y., et al. Protective effects of methylcobalamin, a vitamin B12 analog, against glutamate-induced neurotoxicity in retinal cell culture. Invest Opthalmol Vis Sci, 1997; 38:848-854; Iwasaki, T., Kurimoto, S. Effect of methylcobalamin in accommodative dysfunction of eye by visual load. Sangyo Ika Daigaku Zasshi, 1987; 9:127-132); inhibits HIV-1 infection of normal human blood monocytes and lymphocytes (Weinberg, J. B., Sauls, D. L., Misukonis, M. A., Shugars, D. C. Inhibition of productive human immunodeficiency virus-1 infection by cobalamins. Blood, 1995; 86:1281-1287); and modulates melatonin secretion and normalizes sleep-wake rhythm. (Uchiyama, M., Mayer, G., Okawa, M., Meier-Ewert, K. Effects of vitamin B12 on human circadian body temperature rhythm. Neurosci Lett, 1995; 192:1-4). The content of methylcobalamin in the composition of the present invention may be in the range from 1 mg to 2 g.

In yet another embodiment, the composition of the present invention includes phosphatidylserine (PS), a phosphor-lipid substance produced within the brain. Some studies indicate that PS is highly concentrated in the membrane walls of brain cells, making up about 70% of its nerve tissue mass. It is believed that PS aids in the storage, release, and activity of many vital neurotransmitters and their receptors, thereby aiding cell-to-cell communication. Phosphatidylserine stimulates the release of dopamine, a mood regulator that also controls physical sensations and movement. Also, phosphatidylserine increases the production of acetylcholine, which is necessary for learning and memory, enhances brain glucose metabolism, reduces cortisol levels (the body's central “stress” hormone), and boosts the activity of nerve growth factor (NGF), which is linked to the regulation and health of cholinergic neurons. It has also been shown that aging slows the production of phosphatidylserine to suboptimal levels that, over time, results in diminished mental capacity. An amount of phosphatidylserine added to the composition of the present invention may be in the range from 1 mg to 2 g.

In another embodiment of the present invention, the composition may include glycerylphosphorylcholine (GPC). Studies indicate that GPC has a protective function against age-related brain deterioration and memory loss. Since brain aging is characterized by a cerebral circulatory deficit and a neuro-transmitter deficiency, along with structural deterioration to neurons and their connective transmission lines (axons and dendrites), GPC may be of benefit in interrupting or inhibiting these pathological events. The inventor believes, based on empirical evidence derived from independent neurobiological research, that GPC may be useful in protecting the underlying causes of brain aging while partially restoring cognitive function and improved neuronal signaling. An amount of glycerylphosphorylcholine added to the composition of the present invention may be in the range from 1 mg to 2 g.

In another embodiment of the present invention, the composition may include propentofylline, a glial cell modulator. Propentofylline is one of a family of synthetic versions of xanthine, derived from adenine, one of the four DNA nucleotide bases. With an expanding body of supportive research, it appears propentofylline acts as a nerve growth factor (NGF) synthesis booster, activating NGF production and restoration of mental performance by the initializing of new neuronal connections, following certain types of brain injury or trauma. Additionally, propentofylline provides neuroprotective factors as well in controlling microglia, the inflammatory mechanism that affects neuronal health, neuronal energy deprivation, and free radicals. It has been suggested that Propentofylline may provide therapeutic benefits to patients with Alzheimer-type and vascular-type of dementia (B. Kittner, Use of a combined randomized start/withdrawal design to assess the effects of propentofylline on disease progression in Alzheimer's disease and Vascular dementia, European/Canadian Propentofylline Study Group, New Jersey, US.) An amount of propentofylline added to the composition of the present invention may be in the range from 1 mg to 2 g.

In another embodiment of the invention, the composition may include idebenone. Idebenone is a benzoquinone compound that has been investigated in elderly patients with dementia. Its precise mechanism(s) of action remains unknown, but in vitro and in vivo studies suggest that idebenone may diminish nerve cell damage due to ischaemia, correct neurotransmitter defects and/or cerebral metabolism, and facilitate memory and learning. (Gillis J C, Benefield P, McTavish D. Adis, Drugs Aging 1994 August; 5(2):133-52.) An amount of idebenone added to the composition of the present invention may be in the range from 1 mg to 2 g.

In another embodiment of the invention, the composition may include pyritinol. Recent studies have demonstrated the therapeutic efficacy of pyritinol in patients with senile dementia of the Alzheimer type (SDAT) and multi-infarct dementia (MID). (Fischhof P K, Saletu B, Ruther E, Litschauer G, Moslinger-Gehmayr R, Herrmann W M, Neuropsychobiology 1992; 26(1-2):65-70.) Pyritinol is believed to be effective in promoting overall neurophysiological health, cognitive enhancement, and maintaining integrity of the frontal lobe that is responsible for production of acetylcholine. An amount of pyritinol added to the composition of the present invention may be in the range from 1 mg to 2 g.

In another embodiment of the invention, the composition may include piracetam and its associated or derived “iracetam” drugs, aniracetam, nefiracetam, oxiracetam, pramiracetam and levetiracetam. Piracetam is very similar in molecular structure to the amino acid pyroglutamate (see Pyroglutamate). Piracetam and pyroglutamate have the same “base” chemical structure, the 2-oxo-pyrrolidine, but they differ by the side chain. Pyroglutamate is 2-oxo-pyrrolidine carboxylic acid, and piracetam is 2-oxo-pyrrolidine acetamide. Piracetam enhances cognition under conditions of hypoxia (too little oxygen), and also enhances memory and some kinds of learning in humans. Outside of the U.S., piracetam is used to treat alcoholism, stroke, vertigo, senile dementia, sickle cell anemia, dyslexia, and numerous other health problems (Ward Dean & John Morgenthaler, Smart Drugs & Nutrients). An amount of piracetam and its associated or derived “iracetam” drugs, aniracetam, nefiracetam, oxiracetam, pramiracetam, and levetiracetam added to the composition of the present invention may be in the range from 1 mg to 2 g.

In another embodiment of the invention, the composition may further include hydergine. Hydergine is believed to increase blood supply to the brain (vasodilator), increase oxygen delivered to the brain, enhance metabolism of brain cells, protect the brain from insufficient oxygen supply, slow the deposit of the age pigment lipofuscin in the brain, prevent free radical damage to brain cells, and improve memory, learning and recall. Some European countries use Hydergine for emergencies and accidents that involve shock, hemorrhage, strokes, heart attacks, drowning, electrocution, and drug overdose. Hydergine has also been suggested as an antiaging medicine and an adjunct for the treatment of age-related mental decline. An amount of Hydergine added to the composition of the present invention may be in the range from 1 mg to 2 g.

In another embodiment of the invention, the composition may include galantamine. Galantamine provides a broad range of neuroprotective, central nervous system regulating, and cognitive enhancement features. Efficacy of galantamine in vascular dementia and Alzheimer's disease, combined with cerebrovascular disease, has been demonstrated recently (Erkinjuntti T, Kurz A, Gauthier S, Bullock R, Lilienfeld S, Damaraju, Lancet 2002; 359: 1283-90.) An amount of galantamine added to the composition of the present invention may be in the range from 1 mg to 2 g.

In another embodiment of the invention, the composition may include modafinil (2-[(diphenylmethyl)sulfinyl]acetamide). Modafinil has shown benefits in fatigue-related disorders such as multiple sclerosis and various forms of neurological fatigue. (Cochran J W: Effect of modafinil on fatigue associated with neurological illnesses. J Chronic Fatigue Syndrome 2001; 8:65-70). Current research suggests modafinil may be beneficial in the treatment of Alzheimer's disease, depression, attention-deficit disorder, myotonic dystrophy, age-related memory decline, idiopathic hypersomnia, and everyday cat-napping. An amount of modafinil added to the composition of the present invention may be in the range from 1 mg to 2 g.

In another embodiment of the present invention, the composition may include glutathione (L-gammaglutamyl-L-cysteinylglycine). Glutathione is a tri-peptide of the amino acids cysteine, glycine, and glutamic acid. Glutathione is an antioxidant compound found in living animal and plant tissue. It takes up and gives off hydrogen and is important in cellular respiration. A deficiency of glutathione can cause hemolysis (destruction of red blood cells, leading to anemia) and oxidative stress. Glutathione is essential in intermediary metabolism as a donor of sulfhydryl groups that are essential for the detoxification of acetaminophen. (PDR Medical Dictionary, Spraycar, 1999).

Glutathione prevents oxidative stress in most cells and helps to trap free radicals that can damage DNA and RNA. There is a direct correlation with the speed of aging and the reduction of glutathione concentrations in intracellular fluids. As individuals grow older, glutathione levels drop, and the ability to detoxify free radicals decreases. An amount of glutathione added to the composition of the present invention may be in the range from 1 mg to 2 g.

In another embodiment of the present invention, the composition may include centrophenoxine. Centrophenoxinc is a compound of two biochemicals—dimethylaminoethanol (DMAE) and parachlorophenoxyacetate (pCPA). It is believed that centrophenoxine may be able to deplete age-related lipofuscin accumulation by elevating the activity of antioxidant enzymes. The data also suggest that centrophenoxine may alleviate senescence, possibly by activation of antioxidant enzymes. It is also believed that centrophenoxine may accelerate information processing in the brain and enhance brain cell uptake of glucose. Brain cells use glucose to produce the energy they need to perform their neurological functions and to maintain cell viability. An amount of centrophenoxine added to the composition of the present invention may be in the range from 1 mg to 2 g.

Applicant believes that the efficacy of the above-referenced additional ingredients as well as many other therapeutic compounds may be greatly improved by combining them with the composition of the present invention.

In another aspect, the present invention provides a method of treating at least one neuro-degenerative condition or pathology. The method comprises administering the composition of the present invention to a patient in a single or multiple dosage regimen. In one embodiment, the composition comprises 1 to 50 parts by weight of Selegiline Hydrochloride, 1 to 100 parts by weight of Procaine Hydrochloride, 1 to 50 parts by weight of Vinpocetine, 1 to 2000 parts by weight of the N-GABA ingredient, and 1 to 2000 parts by weight of trimethylglycine. In another embodiment, the composition is administered daily and a daily dose is 1 to 10 mg of Selegiline Hydrochloride, 25 to 75 mg of Procaine Hydrochloride, 1 to 10 mg of Vinpocetine, 1 to 100 mg of the N-GABA ingredient, and 300 to 700 mg of trimethylglycine. In still another embodiment, the daily dose is 2.5 mg of Selegiline Hydrochloride, 50 mg of Procaine Hydrochloride, 5 mg of Vinpocetine, 50 mg of the N-GABA ingredient, and 500 mg of trimethylglycine.

The composition may be administered by a route selected from a group consisting of sublingual infusion, delivery through nasal membranes, injection, oral administration, and transdermal delivery. In one embodiment, the composition is administered via a non-oral route selected from the group consisting of intradermal, subcutaneous, intramuscular, intravenous, intrathecal, sublingual, rectal, vaginal, intraocular, transdermal, respiratory mucosal, and pulmonary routes of administration. In one embodiment, the administering step comprises a weekly cycle of five days administering the composition sublingually and two days off the composition.

The neuro-degenerative condition or pathology may be age-related. The neuro-degenerative condition or pathology may be depression, emotional disturbance, sleep pattern disorder, anxiety disorder, neurosis, neurasthenia, obsessive-compulsive disorder, chronic fatigue syndrome, hyperactivity, panic syndrome, substance dependence, such as a caffeine dependence, or chronic sleep disorder, such as insomnia.

In another aspect, the present invention provides a method of reducing jet lag. The method comprises administering the composition of the present invention to a patient in a single or multiple dosage regimen. In one embodiment, the administration of the composition begins on the day of arrival from a different time zone.

In another aspect, the present invention provides a method of treating inflammatory processes. The method comprises administering the composition of the present invention to a patient in a single or multiple dosage regimen. In one embodiment, the inflammatory process is arthritis and the administering of the composition results in reducing arthritic pain and swelling.

In another aspect, the present invention provides a method of decreasing hormonal imbalance in menopausal and postmenopausal women. The method comprises administering the composition of the present invention to a patient in a single or multiple dosage regimen. It is observed that women treated with the composition of the present invention have a reduction of symptoms of Pre-Menstrual Syndrome (PMS), experience a decrease in chronic internal infections, such as chronic yeast infections, have an improved menstrual cycle regularity. A stabilization of hormonal levels during menopause is also reported.

In another aspect, the present invention provides a method of treating depression symptoms. The method comprises administering the composition of the present invention to a patient in a single or multiple dosage regimen.

In another aspect, the present invention provides a method of improving auditory or visual acuity. The method comprises administering the composition of the present invention to a patient in a single or multiple dosage regimen.

This invention also provides methods of autoimmune enhancement; improving patient's self confidence, self-esteem, social skills, and mental focus; improving aerobic endurance, weight-training capacity, physical agility, and muscle flexibility; treating vascular dementia and Alzheimer's disease; stimulating neuron and nerve cell regrowth; improving blood circulation, reducing blood pressure, and hypertension; treating digestive disorders or improving metabolism; improving sexual performance and libido; stimulating function of the pituitary gland; and increasing a cellular uptake of oxygen and glucose. The methods comprise administering the composition of the present invention to a patient in a single or multiple dosage regimen.

In another aspect, the invention provides a method of increasing concentration of at least one chemical species, such as serotonin, choline, acetylcholine, endorphin, melanocyte-stimulating hormone (MSH), ACTH, dopamine, phenylethylalanine, superoxidase dismutase, catalase, antioxidants, and nitric oxide. In another aspect, the invention provides a method of decreasing levels of cortisol, free radicals, or both. The methods comprise administering the composition of the present invention to a patient in a single or multiple dosage regimen.

In another aspect, the invention provides a method of compensating undesirable side effects associated with individual administration of Selegiline Hydrochloride, Procaine Hydrochloride, Vinpocetine, trimethylglycine, and an N-GABA ingredient selected from a group consisting of N-nicotinyl-gamma-aminobutyric acid (N-GABA), niacin, niacinamide, gamma-aminobutyric acid (GABA). The undesirable side effects may include irritability, hyper-excitability, psychomotor agitation, insomnia, elevated blood pressure levels, heartburn, bloating, dizziness, nausea, headaches, blurred vision, disorientation, tachycardia, and dyspnea.

For the purpose of the present invention, the terms “relieved,” “increased,” “stimulated,” “stimulating,” “mitigated,” “decreased,” “decrease,” “decreasing,” “reduced,” “reducing,” “improving,” “improvement,” “enhancement,” “improvement,” and “compensating” refer to changes associated with a treatment by the composition of the present invention as compared to a pre-treatment state. The changes may be observed and reported by the patient, observed by a physician, and/or measured by a physician. Those skilled in the art would know which analytical methods should be used to measure such changes.

For the purpose of the present invention, the terms “treating” and “substantially eliminating” a condition or a disease mean affecting the condition or the disease in such a way that at least some symptoms associated with the conditions or the disease change when the patient is treated by the composition of the present invention. The changes may be observed and reported by the patient, observed by a physician, and/or measured by a physician.

The following examples are intended to illustrate, but not to limit, the scope of the invention. While such examples are typical of those that might be used, other procedures known to those skilled in the art may alternatively be used. Indeed, those of ordinary skill in the art can readily envision and produce further embodiments, based on the teachings herein, without undue experimentation.

### Example 1

### Preparation of the Composition of the Present Invention for Administration to the Patients

Selegiline Hydrochloride powder was purchased from Medisca Inc. (Plattsburgh, N.Y.). Procaine HCL (C₁₃—H₂₀—N₂—O₂—HCl) powder was purchased from Spectrum Pharmacy Compositions (Tucson, Ariz.). Vinpocetine powder was purchased from Professional Compounding Centers of America (PCCA) (Houston, Tex.). N-GABA was purchased in a form of PICAMILON™ 50 mg tablets from Biologics (New York, N.Y.). In addition to N-GABA, each tablet contained inert ingredients, including calcium phosphate, malt dextrin, magnesium state, and cellulose. Anhydrous Betaine powder was purchased from Spectrum Pharmacy Compositions (Tucson, Ariz.). Avicel PH-105 (microcrystallie cellulose NF) powder was purchased from PCCA (Houston, Tex.).

The PICAMILON™ tablets were ground manually in a pharmacist mortar with a pestle to form a fine microgranulated powder. The remaining ingredients were mixed with the obtained powder in the following order: Selegiline, Vinpocetine, Procaine, Trimethylglycine, and Avicel (inactive ingredient, sweetener). The following amounts of each ingredient were added (in mg):

| | |
|---|---|
| Selegiline Hydrochloride | 2.5 |
| Procaine Hydrochloride | 50 |
| Vinpocetine | 5 |
| N-GABA | 50 |
| Trimethylglycine | 500 |

The obtained composition in the powder form was then placed into individual vials and lids were tightened. Each vial contained 1 g of the composition. The patients were instructed to take the contents of 1 vial on an empty stomach 30 to 45 minutes prior to breakfast by dissolving under the tongue (sublingual infusion). The treatment regimen consisted of weekly cycles of five days of taking the composition and two days off the composition. The cycle was repeated. The patients were asked to drink at least one 8-ounce glass of water immediately after taking the composition and an additional 8-ounce glass of water within the first hour. Also, the patients were asked to avoid caffeine usage for at least 2 to 3 hours after the infusion. A regular diet was suggested. The patients were warned against taking the composition in a combination with antibiotics and/or antidepressants. Some patients observed temporarily and slight side effects such as a runny nose, headache, and increased bowl movements. These side effects disappeared within 3-10 days on the regimen.

### Example 2

### Reduction in Jet Lag and Memory Improvement

A 36-year female patient who has previously complained of severe jet lag associated with both domestic and trans-Atlantic flights was prescribed the composition of Example 1 of the present invention to be taken for 3 days starting on the day of her arrival from Europe. After taking the composition for 3 days, the patient reported that she did not experience jet lag, did not feel tired and fatigued, and had a restful sleep. She also reported that her sugar and coffee cravings greatly decreased and her ability to memorize and focus improved.

### Example 3

### Reduction in Arthritic Pain and Swelling

A 63-year-old female suffering from high blood pressure and severe osteoarthritis was prescribed the composition of Example 1. After 4 weeks on the regimen, the patient's blood pressure decreased from 140/90 to 133/75 and stabilized at 133/75 without the use of blood pressure control medications. After 8 weeks on the regimen, the patient reported a visible decrease in arthritic swelling and pain in her hands.

A 42-year-old male patient suffering from arthritis was prescribed the composition of Example 1. Within one week on the regimen, the patient noted a decrease in the inflammation of joints and the reduction in arthritic pain. He also reported an increased ability to focus, accompanied by improved and more restful sleep.

### Example 4

### Counteracting Hormonal Imbalance

A 39-year-old female patient suffering from Premenstrual Syndrome (PMS) and chronic yeast infections, which had been resistant to conventional treatment prescribed by her physician, was prescribed the composition of Example 1. The patient reported that after 1 week of taking the composition, the yeast infection symptoms disappeared and haven't reoccurred. She also reported that while on the regimen, she did not suffer from any PMS syndromes including cramps, irritability, anxiety, and moodiness. She felt energized and had improved self-confidence and mental concentration. The patient also reported that when she stopped taking the composition for several weeks, her PMS symptoms returned.

A 39-year-old and a 41-year-old female patients treated with the composition of Example 1 reported a reduction in duration of their menstrual cycles. In both cases, menstrual cycles were characterized by increased flow but a shorter duration. In the case of a 39-year-old patient, the cycle was reduced from 5 days to 3 days. In the case of a 41-year-old patient, the cycle was reduced from 6 days to 4 days.

### Example 5

### Treatment of Depression Symptoms

A 44-year-old female patient, who suffered from depression and anxiety, was previously treated with PAXIL® (paroxetine hydrochloride), a selective serotonin reuptake inhibitor (SSRI) and Trazodone HCl, a serotonin 2A antagonist and reuptake inhibitor (SARI). The patient reported that although the medications helped to decrease depression, stress, and anxiety, she suffered from severe side effects, including a loss of short-term memory, constipation, decreased libido, and disturbed sleep. When she stopped taking the medication 6 months later, her depression and anxiety symptoms returned.

After taking the composition of Example 1 for 1 week, the patient reported a high energy level, higher aerobic endurance, fatigue reduction, improved focus, and better sleep. After taking the composition of Example 1 for 5 weeks, the patient reported that her depression and anxiety have disappeared and her libido has greatly improved. She also noted that her eyesight and visual clarity have improved.

### Example 6

### Improvement of Auditory Acuity and Ability to Focus

A 51-year-old male patient was suffering from the loss of hearing acuity in social settings with a substantial amount of background noise, such as restaurants. After 4 weeks on the regimen of Example 1, the patient reported a significant improvement in his audio acuity. In particular, he noted his newly discovered ability to participate in conversations even with a substantial amount of background noise. He also reported an increased ability to focus, improved and more restful sleep, and an increased libido.

### Example 7

### Improvement of Physical Endurance and Agility

An 84-year-old male patient suffered from residual spinal structural weakness and fatigue following surgery for spinal disc fusion preceded by aortic valve replacement. After three weeks on the regimen of Example 1, the patient reported greater overall endurance, particularly significant reduction of pain and lower back disc tension, enabling him to stand for extended periods of time without the need to relieve pressure by sitting. The patient's spinal flexibility and absence of fatigue when moving were also observed by his spouse. The patient further reported overall memory and information recall improvement.

### Example 8

### Treatment of Depression Obsessive/Compulsive Disorder, and Attention Deficit Hyperactive Disorder (ADHD)

A 31-year-old male patient was diagnosed in 1993 with moderate depression, OCD-obsessive/compulsive disorder, and ADHD. He was first prescribed Prozac for 14 months followed by Wellbutrin. Then, he was represcribed Prozac, then Zoloft for two months, followed by Paxil for two years, followed by Ritalin for two months. The side effects experienced by the patient included significant decrease in sex drive, difficulty in maintaining erection (first encountered at age 25 on Prozac, as dosing was titrated up from 20 mg to 60 mg daily). Side effects on Wellbutrin included paranoia and acute panic symptoms.

Conjunctive use of Prozac and Wellbutrin was attempted to mitigate lack of sexual desire and erectile dysfunction disorder, but the two combined antidepressants failed to remedy the problem. Patient's physician then prescribed Paxil for 8 months, which resulted in rapid weight gain, increased aggressiveness, abnormally intense cravings for alcohol, decreased sex drive, difficulty in maintaining erections, general listlessness, and no desires to initiate or accomplish anything either in the patient's personal or professional life. After patient was prescribed Ritalin, general listlessness continued along with general malaise and lack of focus.

After 4 weeks on the regimen of Example 1, patient reported a significant elevation of mood, reduction of obsessive/compulsive behavior, which previously manifested itself in substantial daily intake of caffeine compositions and indulgence in high fat and high sugar snack foods. In the fifth week, patient felt no further withdrawal symptoms from a significant reduction in daily coffee intake and reduced fat and sugar diet.

During the sixth to eighth week, the patient felt increased, sustainable energy, mood elevation, mental acuity, focus and renewed sense of purpose, both personally and professionally. The patient reported no adverse side effects. After 5 months on the regimen, the patient reported that he is substantially free from symptoms of depression.

It will be apparent to those skilled in the art that various modifications and variations can be made in the compositions and methods of the present invention without departing from the spirit or scope of the inventions. Thus, it is intended that the present invention cover modifications and variations of this invention that come within the scope of the appended claims and their equivalents.

What is claimed is:

1. A composition comprising:
1 to 10 parts by weight of Selegiline Hydrochloride;
1 to 100 parts by weight of Procaine Hydrochloride;
1 to 10 parts by weight of Vinpocetine, 1 to 2000 parts by weight of trimethylglycine; and
1 to 100 parts by weight of an N-GABA ingredient selected from the group consisting of N-nicotinyl-gamma-aminobutyric acid (N-GABA), niacin, niacinamide, gamma-aminobutyric acid (GABA), and combinations thereof,
wherein the composition comprises such amounts of Selegiline Hydrochloride, Procaine Hydrochloride, Vinpocetine, the N-GABA ingredient, and trimethylglycine that undesirable side effects associated with administration of Selegiline Hydrochloride, Procaine Hydrochloride, Vinpocetine, N-GABA ingredient, or trimethylglycine on its own are substantially eliminated.

2. The composition of claim 1 comprising 25 to 75 parts by weight of Procaine Hydrochloride and 300 to 700 parts by weight of trimethylglycine.

3. The composition of claim 1 comprising 2.5 parts by weight of Selegiline Hydrochloride, 50 parts by weight of Procaine Hydrochloride, 5 parts by weight of Vinpocetine, 500 parts by weight of trimethylglycine, and 50 parts by weight of the N-GABA ingredient.

4. The composition of claim 1, wherein an amount of Selegiline Hydrochloride, Procaine Hydrochloride, Vinpocetine, trimethylglycine, and the N-GABA ingredient is such that when the composition is administered to a patient, at least one health condition is relieved, wherein the health condition is selected from the group consisting of anxiety, hyperactivity, panic symptoms, fatigue, hypertension, hormonal disbalance, jet lag, substance dependence, insomnia, loss of appetite, and emotional disturbance.

5. The composition of claim 4, wherein the substance dependence is caffeine dependence.

6. The composition of claim 4, wherein the hormonal disbalance is a hormonal disbalance in menopausal and post-menopausal women.

7. The composition of claim 1, wherein an amount of Selegiline Hydrochloride, Procaine Hydrochloride, Vinpocetine, trimethylglycine, and the N-GABA ingredient is such that when the composition is administered to a patient, a concentration of at least one chemical species is increased, wherein the chemical species is selected from the group consisting of serotonin, choline, acetylcholine, endorphin, melanocyte-stimulating hormone (MSH), ACTH, dopamine, phenylethylalanine, superoxidase dismutase, catalase, antioxidants, and nitric oxide.

8. The composition of claim 1, wherein an amount of Selegiline Hydrochloride, Procaine Hydrochloride, Vinpocetine, trimethylglycine, and the N-GABA ingredient is such that when the composition is administered to a patient, a function of the pituitary gland is stimulated.

9. The composition of claim 1, wherein an amount of Selegiline Hydrochloride, Procaine hydrochloride, Vinpocetine, trimethylglycine, and the N-GABA ingredient is such that when the composition is administered to a patient, symptoms of at least one disease or disorder are mitigated, wherein the disease or disorder is selected from the group consisting of depression, sleep pattern disorder, anxiety disorder, neurosis, neurasthenia, obsessive-compulsive disorder, chronic fatigue syndrome, digestive diseases, hypertension, ostcoarthritis, and loss of libido.

10. The composition of claim 1, wherein an amount an amount of Selegiline Hydrochloride, Procaine Hydrochloride, Vinpocetine, trimethylglycine, and the N-GABA ingredient is such that when the composition is administered to a patient, a cellular uptake of oxygen and glucose is increased.

11. The composition of claim 1, wherein an amount of Selegiline Hydrochloride, Procaine Hydrochloride, Vinpocetine, trimethylglycine, and the N-GABA ingredient is such that when the composition is administered to a patient, levels of at least one chemical species are reduced, wherein the chemical species is selected from the group consisting of cortisol and free radicals.

12. The composition of claim 1, wherein the undesirable side effects are selected from the group consisting of irritability, hyper-excitability, psychomotor agitation, insomnia, elevated blood pressure levels, heartburn, bloating, dizziness, nausea, headaches, blurred vision, disorientation, tachycardia, and dyspnea.

13. The composition of claim 1, wherein the amount of Selegiline Hydrochloride, Procaine Hydrochloride, Vinpocetine, trimethylglycine, and the N-GABA ingredient is such that when the composition is administered to a patient, at least one neuro-endocrine or physiological improvement is achieved.

14. The composition of claim 13, wherein the neuro-endocrine or physiological improvement is selected from the group consisting of increased self-confidence, increased self-esteem, elimination of the need for anti-depressants, cognitive enhancement, improvement of aerobic endurance, improvement of weight-training capacity, improvement of blood circulation, reduction of blood pressure, improvement of sexual performance, improvement of libido, improvement of visual acuity, improvement of auditory acuity, improvement of physical agility, improvement of muscle flexibility, improvement of social interaction skills, promotion of relaxation response, reduction of arthritic pain and swelling, enhancement of immune system functioning, improvement of metabolism, and improvement of skin tone.

15. The composition of claim 14, wherein the improvement of metabolism is a relief from constipation.

16. The composition of claim 14, wherein the cognitive enhancement comprises improvement of at least one mental ability selected from the group consisting of concentration, clarity, alertness, and mental focus.

17. The composition of claim 1, wherein the composition is in a form of a solid, liquid, or aerosol.

18. The composition of claim 1, wherein the composition is in a form of an oral tablet, capsule, powder, a nebulized vapor, or a transdermal patch.

19. The composition of claim 1, wherein the composition is in the form of a powder.

20. The composition of claim 1 further comprising at least one formulation additive selected from the group consisting of water, an alcohol, an amylaceous substance, a thickener, a fatty acid, a flavor, a coloring composition, a sweetener, a seasoning, a separator, a preservative, a carrier, an excipient, an adjuvant, a diluent, and a stabilizer.

21. The composition of claim 20, wherein the separator is a microcrystalline a-cellulose.

22. The composition of claim 1 further comprising at least one compound selected from the group consisting of a therapeutic compound, a nutrition supplement, a vitamin, an herb, a homeopathic composition, a prescription medication, a mineral, and a trace element.

23. The composition of claim 22, wherein the therapeutic compound is selected from the group consisting of hematoporphyrin, acetyl-L-carnitine, methylcobalamin, methylcobalamin, glycerylphosphorylcholine, propentofylline, idebenone, pyritinol, piracetam, aniracetam, nefiracetam, oxiracetam, pramiracetam, levetiracetam, hydergine, glutathione, modafinil, centrophenoxine, and galantamine.

24. The composition of claim 22, wherein each therapeutic compound is added in an amount from 1 mg to 2 g.
